(19) United States
(12) Patent Application Publication
(10) Pub. No.: US 2007/0270583 A1
(43) Pub. Date: Nov. 22, 2007

(54) Process for Purification of 6 Acetyl 4,1', 6' Trichlorogalactosucrose and 4,1', 6' Trichlorogalactosucrose by Chromatography on Silanized Silica Gel

(75) Inventors: Rakesh Ratnam, Maharashtra (IN); Sundeep Aurora, Maharashtra (IN)

Correspondence Address: NATH & ASSOCIATES, 112 South West Street, Alexandria, VA, 22314, US

(73) Assignee: PHARMED MEDICARE PRIVATE LIMITED, Maharashtra (IN)

(21) Appl. No.: 11/792,621

(22) PCT Filed: Dec. 9, 2005
(86) PCT No.: PCT/IN05/00409

§ 371 Date: Jun. 8, 2007

(30) Foreign Application Priority Data
Dec. 10, 2004 (IN) 1317/MUM/2004

(51) Int. Cl. C07H 1/06

(52) U.S. Cl. 536/127

(57) ABSTRACT

A reverse phase column chromatographic process of purification of 6-acetyl-4,1′,6′trichlorogalactose and 4,1′,6′trichlorogalactose is described which uses silanized silica as stationary phase and water or predominatly aqeous mixture derived from water and small proportion of organic solvents as a mobile phase.

### TECHNICAL FIELD

The present invention relates to a process and a novel strategy for synthesis of chlorinated sucrose, 1′-6′-Dichloro-1′-6′-DIDEOXY-β-Fructofuranasyl-4-chloro-4-deoxy-galactopyranoside.

### BACKGROUND OF INVENTION

Chlorinated sucrose preparation is a challenging process due to the need of chlorination in selective less reactive positions in sucrose molecule in competition with more reactive positions. Generally, this objective is achieved by a procedure which involves essentially protecting the hydroxy group in the pyranose ring of sugar molecule by using various protecting agents such as alky/aryl anhydride, acid chlorides, orthoesters etc., and the protected sucrose is then chlorinated in the desired positions (1′-6′ &, 4) to give the acyl or aryl ester derivative of the product, which is then deesterified to give the desired product 1′-6′-Dichloro-1′-6′-DIDEOXY-β-Fructofuranasyl-4-chloro-4-deoxy-galactopyranoside i.e. 4,1′,6′trichlorogalactosucrose (TGS).

Strategies of prior art methods of production of TGS are based on following: Sucrose-6-acetate is chlorinated by Vilsmeier-Haack reagent to form 6-acetyl-TGS. After chlorination, the deacetylation of 6-acetyl-TGS to

TGS is carried out in the reaction mixture itself. The TGS is then purified from the reaction mixture in various ways based on selective extraction into water immiscible solvent or solvents. The product obtained is finally purified in various ways including column chromatography on silica gel.

Use of “normal phase” column chromatography, i.e. where adsorbent is polar and desorbent is non-polar, for liquid phase separation of reaction mixture containing TGS, 6-acetyl-TGS and process streams including reaction mixtures derived from production process of TGS through chlorination route is well known in prior art.

Mufti et al. (1983) in patent no. U.S. Pat. No. 4,380,476 has described separation of sucrose-6-acylates from reaction mixture by column chromatography on silica gel as well as ion exchange resins including polystyrene resin when they disclosed that “As stated above in the general definition of the process of the invention, the separation of the required 6-acylate from other acylates can be effected before or after chlorination. Most preferably, the initial mixture of acylates obtained from step (a) is separated in step (b) to give a fraction which consists of, or is rich in, the required 6-acylate. This separation can be effected by chromatography, for example on silica gel. However, it is a preferred feature of the present invention, that the separation in step (b) is effected by ion exchange resin chromatography. Any suitable ion exchange resin may be used, and the art of separation of saccharides on such resins is well documented. A polystyrene sulphonic acid cation exchange resin is particularly suitable, . . . Dow Chemical Company”.

In the same patent, Mufti et al. (1983) go further to describe that “Alternatively, the success of the overall process according to the present invention will depend in part on the fact that TGS itself can be isolated without undue difficulty from the deacetylated mixture of chlorinated sucrose derivatives obtained. We have found that chromatography, e.g. on silica gel, will isolate TGS relatively simply. For example, elution of the deacylated mixture with a series of eluants of increasing polarity removes first the less polar by-products and then TGS, while more polar compounds remain bound. Mixtures of chloroform and acetone are particularly suitable: a 2:1 mixture followed by a 1:1 mixture is effective in isolating TGS in the 1:1 eluate. We prefer to chromatograph after deacylation, but chromatographic separation of TGS 6-acylate is also possible”, The pattern of elution described here, in the prior art, is exactly reverse of what we are reporting here in our invention which involves reverse phase chromatography of on silanized silica gel the first elution is of the most polar impurity, followed by the TGS and the less polar impurities remain bound”.

Mufti et al. (1983) further describe in example 1 that “The mixture was concentrated and eluted from a column of silica gel (Merck Kieselgel 60 70-230 mesh ASTM, approx 75 g) using (chloroform:acetone; 2:1) initially and then (chloroform:acetone; 1:1) as eluent giving TGS in approximately 15% yield overall from the starting sucrose.” and in example no.3 that “After de-esterification of the mixture, TGS was isolated by chromatography on silica gel (as before).”

Rathbone et al. (1989) in U.S. Pat. No. 4,826,962 have also mentioned use of chromatographic process for direct isolation of sucralose in the specification as “The separation of the sucralose product may be achieved by any convenient steps, for example by evaporation and extraction into an organic solvent, by chromatographic techniques, or by selective crystallization from either the aqueous or the non-aqueous systems.” and in example 4 as “The products were separated by chromatography and, in addition to sucralose, the presence of 6-chlorogalactose and TCR was detected”.

Catani et al. (1999) in U.S. Pat. No. 5,977,349 have claimed chromatographic purification of chlorinated sucrose. They have given examples of use of sodium sulphonic acid and silica gel for column chromatographic separation of reaction mixtures in liquid phase derived from process streams of production of chlorinated sucrose as described by Walkup et al. (1990) U.S. Pat. No. 4,980,463 which can be subjected to steam stripping and Navia et al. (1996) U.S. Pat. No. 5,530,106. However, the language of the patent is not worded in wording clear enough and it does not make clear which is the novelty claimed and what is the inventive step. As discussed above, the concept of use of column chromatography with silica gel and/or ion exchange resins for separation and purification of constituents of the liquid reaction mixture streams containing chlorinated sucrose derivatives is not novel because it is a prior art, which has also been described very elaborately by Mufti et al. (1983) and Rathbone et al. (1989). Catani et al. (1999) do claim about the “first chlorinated sucrose” without specifying whether it refers to a specific chlorinated sucrose or just “any” of the chlorinated sucrose derivatives, since it actually points out to “any” of the chlorinated sucrose derivatives that may enter the chromatographic column first and elute out of the column first, it actually provides a generic description of scientific principle on which separation is achieved in adsorption column chromatography in general and does not claim or describe any patentable novelty nor any inventive step.

Thus, in all the prior art processes involving “normal phase” chromatography, conventional silica gel, sodium sulphonic acid or polystyrene resin are used as adsorbent and eluents are one of or a mixture of organic solvents including ethyl acetate, methanol, methylene dichloride, ethylene dichloride, acetone, etc., in varying compositions.

However, this method of column chromatography is very expensive as large volume of expensive solvents are required to be used as mobile phase, which need to be recovered from the column fractions by equally expensive distillation.

A less expensive process of column chromatographic purification was needed.

### SUMMARY OF INVENTION

In an improvement over this conventional “normal phase” process, in the invented process, “reverse phase” chromatography was used, wherein silica gel was coated/silanized using commercially available silanes such as trichloromethylsilane, dimethyldichlorosilane, trimethylchlorosilane, triethylchlorosilane, etc. These silanes were used either individually or in combinations to give different grades of hydrophobic silica.

The silanized silica was then taken as stationary phase for column purification where water or buffered aqueous solutions or combination of water miscible solvents such as methanol, acetone, acetonitrile, etc were used with water as mobile phase.

This invention also covers in its scope variations and adaptations of above method within the scope of reverse phase chromatography including use of non-polar adsorbents other than silanized silica also.

### BRIEF DESCRIPTION OF DRAWINGS

FIG. 1: Describes the flow chart of purification of 6-acetyl,4,1′,6′-trichlorogalacto sucrose on silanized silica gel.

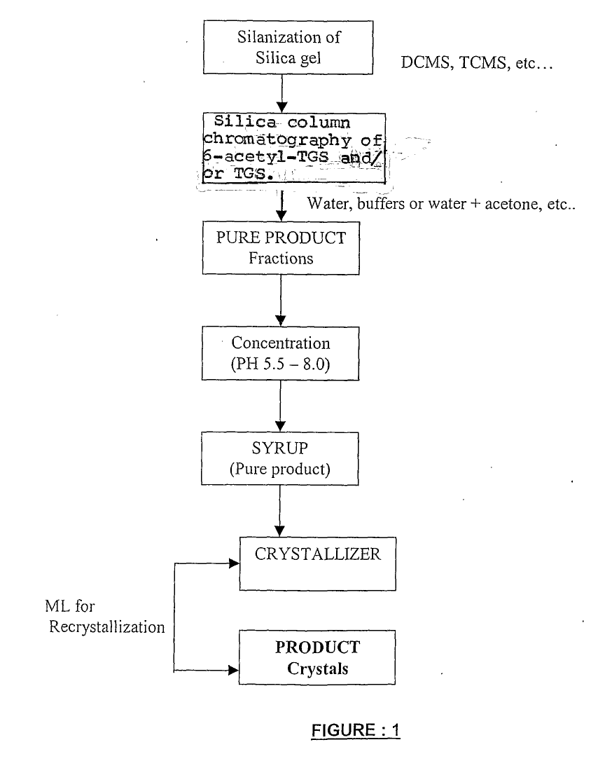

FIG. 2: Various fractions obtained after elution of silanized silica gel column by aqueous eluants.

### DETAILED DESCRIPTION OF INVENTION

Various methods of silanization of silica gel are reported in United States Pharmacopoea, and (X. S. Zhao and G. Q. Iu,1998, J. Phys. Chem. B 1998, 102, 1556-1561). They included the following:

Silanization of silica gel is carried out by allowing the vapors of the silanating agent such as trimethylchlorosilane, dimethyidichlorosilane to coat on to the silica gel in a closed environment. This process takes long hours usually between 6 to 48 hours. After the silanization, the silica is dispersed in water and the Silanized silica gel floats at the top of the solution. This silica is skimmed off and dried before usage in chromatography.

Other alternative ways of silanization are reported to be carried out in the presence of solvents such as toluene, Xylene, ethylene dichloride, etc. Silica gel is suspended in toluene and appropriate amount of the silanating agent is added usually between 1:0.2 to 1:3 times (W/W) of silica gel and heated to 40-45° C. and then filtered and washed with methanol and water. Column chromatographic separation on silanized silica gel is applicable for purification of a number of compounds including 6-acetyl-TGS as well as TGS from reaction mixtures or from solutions done for any purpose.

As mobile phase, water or buffered aqueous solutions were used for elution of the desired product. Also combination of water miscible solvents such as methanol, acetone, acetonitrile, etc were used from 2-8% most preferably 3-5% with water. The pattern of elution of impurities and product was in reverse as compared to the use of the hydrophilic silica as stationary phase in conventional methods of “normal phase” type. This method of column chromatography gave the following advantages:

a) Faster and better elution of product

b) Better re-usability of stationary phase

c) Dramatic cost reduction of elution (solvent) of product fractions

The Silanized silica gel also has the advantage of recycling capability more than the normal phase silica gel and therefore cost of silanization is easily absorbed in it.

On industrial scale, column chromatography is performed in variety of different ways described below to increase the efficiency. Adaptation of method of this invention to all the known different ways of column chromatography on large scale are included within the scope of this specification.

Various such techniques include one or more of following:

a) Fixed bed adsorbent contained with in a column, wherein the feed and mobile phase is injected at one end, they follow an axial traverse separation and fractions get collected at other end

b) Fixed bed adsorbent is contained within a column, the feed and mobile phase are injected at the circumference, they follow a radial traverse separation and get collected at the inner channel at the center.

c) Fixed bed adsorbent contained in a column, the feed and mobile phase being injected through the inner channel at the center, they follow the radial traverse separation and column fractions get collected at the circumference

d) Fixed bed of solid adsorbent in a vertically mounted rotating annulus, the feed and mobile phase being injected at the top and the separated column fractions are collected at the bottom

e) Fixed bed of solid adsorbent contained within several serial sections/columns in closed loop, each individually capable of receiving and relieving fluid with fixed arrangement of feed, desorbent and take off ports, that ratchet forward at fixed intervals in a direction concurrent with the liquid flow, simulating counter current movement of the fixed bed adsorbent.

There could be several variations of above improved techniques and new techniques may also come up in future. All analogous techniques meant for improvement in efficiency of column chromatography are included within the scope of this patent if reverse phase chromatography is used in general and silanized silica gel is used as adsorbent in particular when solution chromatographed contains chlorinated sucrose derivatives or their precursors or derivatives including 6-acetyl-TGS or TGS.

The composition of matter to be chromatographed in this invention can come either as a solution of 6-acetyl-TGS or TGS prepared in water or suitable solvents or as a process stream from a process of production of 6-acetyl-TGS or TGS. The said process of production of 6-acetyl-TGS or TGS includes Mufti et al. (1983) U.S. Pat. No. 4380476, Walkup et al. (1990 U.S. Pat. No. 4,980,463), Jenner et al. (1982) U.S. Pat. No. 4,362,869, Tulley et al. (1989) U.S. Pat No. 4,801,700, Rathbone et al. (1989) U.S. Pat No. 4,826,962, Bornemann et al. (1992) U.S. Pat No. 5,141,860, Navia et al. (1996) U.S. Pat. No. 5,498,709, Simpson (1989) U.S. Pat. No. 4,889,928, Navia (1990) U.S. Pat No. 4,950,746, Neiditch et al. (1991) U.S. Pat No. 5,023,329, Walkup et al. (1992) U.S. Pat. No. 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat No. 5,449,772, Sankey et al. (1995) U.S. Pat. No. 5,470,969, Navia et al. (1996) U.S. Pat. No. 5,498,709, Navia et al. (1996) U.S. Pat. No. 5,530,106 and patent applications containing similar patentable matter including in co-pending application Nos. WO 2005/090374 A1 and WO 2005/090376 A1.

In the following description are given some examples to illustrate basic working of this invention. The reactants used, proportions of reactants used and conditions of chromatography given in the example are only illustrative and are not to be construed to limit the scope of this specification in any way. Any reasonable modifications in the methods described which is obvious to a person skilled in the art, are of analogous and generic in nature, are construed to be within the scope of this invention.

Anything described in singular includes its plural also, unless contrary to the context, viz: when “A process of purification ______ ” is mentioned, it encompasses and covers within its scope, the process expressly mentioned, if any, as well as any or several other known processes of purification also; when “A process of purification including column chromatography” is mentioned, it encompasses and covers within its scope several other known processes of purification, one of which is column chromatography.

The product fractions obtained after chromatography were pooled together and concentrated. During concentration, pH was not allowed to fall less than 5.5 and not go higher than 8.0. The concentrated syrup was allowed to crystallize. The crystallized products were centrifuged or filtered. In case of 6-acetyl-TGS, the pooled fractions were either deacetylated before or after concentration or crystallized out as described above and were stored for future use. The product TGS was tested for the required specifications, milled and packed.

### EXAMPLE 1

Preparation of Silanized Silica Gel

200 kg of silica gel (230 to 400 mesh size) was taken slurried in 400 L of toluene in a glass lined reactor. The uniform slurry was prepared in the reactor with constant mixing for 30 minutes, following which 100 L of trimethylchlorosilane was added. The mixture was mixed thoroughly at slightly elevated temperature of up to 45° C. After 2 hours of stirring, the silica gel was filtered through the nutsche filter and the mother liquour was collected separately. The silica gel cake obtained was washed with 200 L of methanol thoroughly to remove toluene traces and washed with water.

15 kg of sucrose-6-acetate was taken for chlorination. The Vilsmeier-Haack reagent was prepared by taking 63 kg of PCl₅ and 255 L of DMF in glass lined reactor. Then the Vilsmeier-Haack reagent formed was cooled to 0° C. and then the sucrose-6-acetate was added with continuous stirring Then the reaction mixture was allowed to come to room temperature and then heated to 80° C., maintained for 3 hours, further heated to 100° C. and maintained for 6 hours. Then the reaction mixture was heated up to 115° C. and maintained for 90 minutes and then neutralized with water and calcium hydroxide up to pH 7.5. The neutralized mass containing the 6-acetyl TGS was then subjected to rapid drying at controlled temperatures in ATFD (Agitated Thin Film Dryer) (as described in details in co-pending patent application nos. in co-pending application Nos. WO 2005/090374 A1 and WO 2005/090376 A1).

The ATFD powder thus obtained was then dissolved in 1:3 times of volume of water and the pH was adjusted to neutral and extracted into 1:3 times of volume of ethyl acetate. The ethyl acetate was then distilled off under vacuum and then the syrup obtained was mixed with 1:3 times v/v of methanol and was deacetylated using calcium hydroxide at pH 9.5. After deacetylation, the mass was neutralized with 4% Sulphuric acid and methanol was evaporated off. The syrup thus obtained was taken for column chromatographic purification.

100 kg of Silanized silica was slurried in methanol and packed in a SS column (300 mm×3150 mm). The silica was allowed to stand in the column for 16 hours for settling and methanol was flushed out by gravity and as the methanol passed out through the bottom end of the column, 0.05 molar sodium acetate buffer at pH 9.0 was passed through the column and was flushed out till complete removal of methanol. The eluent from the column was checked for methanol content by GC and was ascertained to be less than 1%.

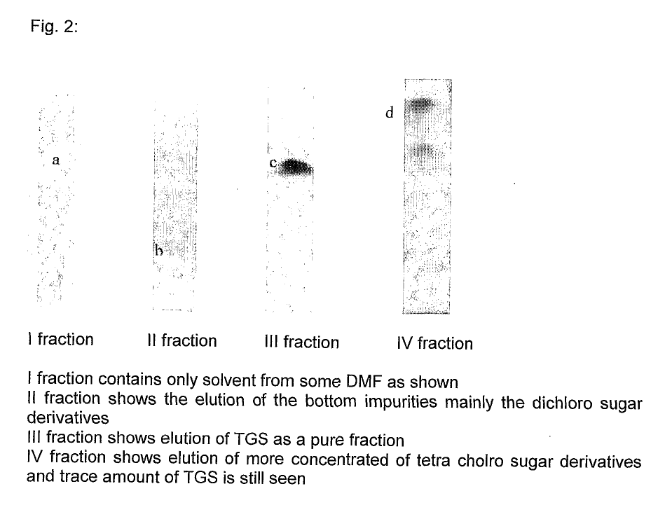

12 kg of crude product syrup (containing 19% of TGS) was loaded on to the column at the top end and was allowed to pass through the silica gel by applying slight pressure by using a metering pump or nitrogen air pressure. As the product syrup was injected through the top end of the column, the same was followed by the buffer at pH 9.0 (0.05 molar sodium acetate). The fractions collected from the bottom of the column were periodically checked for impurities and TGS content. The flow rate was adjusted to 120 ml/min. Elution pH is 6.3-6.5. Fractions are collected after the loading of TGS to the column. A typical pattern of TLC of fractions is in FIG. 2.

A profile of the different fractions collected is given in Table 1 below:

TABLE 1

| | TGS in eluent |
|---|---|
| Fractions & volume | solution (gms) |
| 0 L to 100 L | 0 g |
| 100 L to 135 L | 22 g |
| 135 L to 160 L | 55 g |
| 160 L to 280 L | 1860 g |
| 280 L to 295 L | 280 g |
| 295 L to 335 L | 26 g |
| 335 L to 400 L | 3.5 g |

Pure fractions as confirmed by TLC and HPLC were pooled and concentrated by reverse osmosis membrane system. The concentrated fraction containing 1.86 kg of TGS was extracted into 1:3 times volume of ethyl acetate and further concentrated and crystallized.

### EXAMPLE 2

12 kg of sucrose-6-acetate was taken for chlorination. The Vilsmeier-Haack reagent was prepared by taking 50 kg of PCl₅ and 255 L of DMF in glass lined reactor. Then the Vilsmeier-Haack reagent formed was cooled to 0° C. and then the sucrose-6-acetate was added with continuous stirring. Then the reaction mixture was allowed to come to room temperature and heated to 80° C., maintained for 3 hours, further heated to 100° C. and maintained for 6 hours. Then the reaction mixture was heated up to 115° C. and maintained for 90 minutes and then neutralized with water and calcium hydroxide up to pH 7.5. The neutralized mass containing the 6-acetyl TGS was then subjected to ATFD for DMF removal.

The ATFD powder thus obtained was then dissolved in 1:3 times of water and the pH was adjusted to neutral and extracted into 1:3 times of ethyl acetate. The ethyl acetate was then distilled off under vacuum and then the syrup obtained was taken for column chromatographic purification.

100 kg of Silanized silica was slurried in methanol and packed in a SS column (300 mm×3150 mm). The silanized silica was allowed to stand in the column for 16 hours for settling and methanol was flushed out by gravity and as the methanol passed out through the bottom end of the column, 0.05 molar sodium acetate buffer at pH 9.0 was passed through the column and was flushed out till complete removal of methanol. The eluent from the column was checked for methanol content by GC and was ascertained to be less than 1%.

18 kg of crude product syrup (containing 15% of 6-acetyl-TGS) was loaded on to the column at the top end and was allowed to pass through the silica gel by applying slight pressure by using a metering pump or nitrogen air pressure. As the product syrup was injected through the top end of the column, was followed by the buffer at pH 9.0 (0.05 molar sodium acetate). The fractions collected from the bottom of the column were periodically checked for impurities and TGS content. The flow rate was adjusted to 120 ml/min. Elution pH is 6.3-6.5. A profile of the different fractions collected is given in Table 2 below. Fractions are collected after the loading of TGS to the column

TABLE 2

| | 6-acetyl TGS in |
|---|---|
| Fractions & volume | eluent solution (gms) |
| 0 L to 100 L | 0 g |
| 100 L to 140 L | 28 g |
| 140 L to 175 L | 70 g |
| 175 L to 310 L | 2360 g |
| 310 L to 345 L | 180 g |
| 345 L to 390 L | 29 g |
| 390 L to 450 L | 12 g |

Pure fractions as confirmed by TLC and HPLC were pooled together and the pH was adjusted to 9.5 and was stirred in a SS reactor and deacetylation was monitored by TLC. After deacetylation, the pH of the solution was bought to neutral by adding dilute HCl and concentrated by reverse osmosis membrane system.

The concentrated fraction containing 2.3 kg of TGS was extracted into 1:3 times volume of ethyl acetate and further concentrated and crystallized.

### EXAMPLE 3

An experiment was conducted to compare between the use of normal silica gel and Silanized silica gel for column chromatographic purification of TGS. The crude quantity of mixture of reactants subjected for chromatography was 100 kg (15 kg of TGS). Two columns (750 mm×3150 mm) were taken for the experiment, one for loading with normal silica gel and other for leading with Silanized silica gel. Each of the column was packed with 800 kg of the respective silica gel. The normal silica was eluted with varying proportions of ethyl acetate and methylene dichloride as mobile phase and the Silanized silica was eluted with pH 9.0 sodium acetate buffer. The different fractions obtained and their profiles are shown in Table 3 below:

TABLE 3

| Normal silica gel | TGS in eluent | Silanized silica gel | TGS in eluent |
|---|---|---|---|
| Fractions volume | (kg) | Fractions volume | (kg) |
| 0 L to 550 L | 0 g | 550 L | 0 g |
| 550 to 1250 L | 0.23 kg | 550 L to 700 L | 0.12 kg |
| 1250 L to 1750 L | 0.63 kg | 700 L to 850 L | 4.4 kg |
| 1750 L to 2250 L | 2.36 kg | 850 L to 1100 L | 4.97 kg |
| 2250 L to 2750 L | 3.69 kg | 1100 L to 1350 L | 3.65 kg |
| 2750 L to 3250 L | 3.76 kg | 1350 L to 1600 L | 1.56 kg |
| 3250 L to 3750 L | 3.36 kg | 1600 L to 1850 L | 0.083 kg |
| 3750 L to 4250 L | 0.68 kg | | |
| 4250 L to 4750 L | 0.08 kg | | |

After the elution of fractions, the column was flushed with 100 L of methanol followed by 200 L of pH 9.0 sodium acetate buffer and was reused again. This flushing of silica gel after every batch of column loading is mandatory in order to reactivate the silica gel. The Silanized silica gel can be used for 10 to 15 such batches without regeneration. This is not in the case of normal silica gel, if the flushing is to be carried out in the column itself, it would consume a large volume of organic solvents. Hence the silica gel is unloaded from the column after every batch and is regenerated and then repacked. The whole process is time consuming and very cumbersome when compared to using the Silanized silica gel.

A typical comparison of the usage of normal phase silica gel column chromatography and reverse phase silica gel column chromatography is shown below in table no. 4. The basis is the stationary phase quantity is 500 kg and the TGS to be separated is equivalent to 13-15 kg.

TABLE 4

| Solvent used as | | Cost of solvent | Cost of aqueous |
|---|---|---|---|
| mobile phase in | Aqueous buffer | per kg of TGS | buffer per kg of |
| normal phase | solution used in | purified by | TGS purified by |
| chromato- | Silanized silica | normal phase | Silanized silica |
| graphy | chromatography | chromatography | chromatography |
| 5000 L | 1800 L | US $37 at 10% | US $0.4 |
| | | consumption of | |
| | | solvent | |

The figures of price given above shall change based on criteria used for costing. However, they do give the general idea on kind of difference that will be expected to occur in most of the perspectives of costing. It is clear that there shall be substantial saving of costs in use of column chromatography on silanized silica gel.

1. A process of purification, including a process of column chromatography, of chlorinated sucrose, their precursors and derivatives from aqueous compositions containing them, wherein
a. adsorbent is non-polar including silica gel silanized by a method comprising silane vapour pressure coating on silica gel or silanizing silica gel in a solvent comprising toluene, xylene, benzene, ethylene dichloride, methylene dichloride etc.
b. desorbent used is polar comprising one or more of (a) water, or (b) a predominantly aqueous solution comprising buffered solutions at various pH preferably between 7 to 10, or (c) water with acetonitrile or acetone preferably in 5% concentration v/v, or (d) methanol in water preferably 2% concentration v/v, or (e) an organic solvent miscible in water in any ratio.

2. A process of claim 1 wherein the said chlorinated sucrose includes 4,1′,6′trichlorogalactosucrose (TGS) and the said precursor or derivative of chlorinated sucros includes 6-acetyl-4,1′,6′trichlorogalactose (6-acetyl-TGS).

3. A process of claim 2 wherein the said aqueous composition subjected to chromatographic separation is derived from in one or more of following means:
a. dissolution of the TGS or 6-acetyl-TGS, in an aqueous medium
b. as a process stream, an aqueous reaction mixture, derived from a process of production of TGS or 6-acetyl-TGS.
